(19) United States
(12) Patent Application Publication
(10) Pub. No.: US 2015/0320372 A1
(43) Pub. Date: Nov. 12, 2015

(54) RADIATION IMAGING APPARATUS

(71) Applicant: CANON KABUSHIKI KAISHA, Tokyo (JP)

(72) Inventors: Genta Sato, Kawasaki-shi (JP); Hidetoshi Tsuzuki, Yokohama-shi (JP)

(21) Appl. No.: 14/697,793

(22) Filed: Apr. 28, 2015

(30) Foreign Application Priority Data
May 12, 2014 (JP) 2014-098835

(51) Int. Cl. A61B 6/00; G21K 1/10; G21K 1/06; G01N 23/20

(52) U.S. Cl. CPC A61B 6/4035; A61B 6/5282; A61B 6/5235; G01N 23/20075; G21K 1/10; G21K 1/067

(57) ABSTRACT

A radiation imaging apparatus includes: a beam splitter; a detector that detects radiation passing through the beam splitter and the object; and a processing device that generates image data representing information on an inside of the object on the basis of data obtained with the detector. A first distance which is a distance between the beam splitter and the object can be changed. The processing device has a determination unit that determines the first distance that is to be set when the object is imaged, such that a value of the information on the inside of the object falls within a dynamic range of the image data.

### BACKGROUND OF THE INVENTION

1. Field of the Invention

The present invention relates to a radiation imaging apparatus.

2. Description of the Related Art

Imaging apparatuses for imaging internal information on an object by using radiation, primarily X-rays, are used for multiple purposes in the field of medical diagnosis and non-destructive testing. In recent years, a method for generating an image relating to phase modulation or scattering intensity induced by an object from changes (distortions) of the radiation intensity pattern that has passed through the object has attracted attention. This method is called radiation phase imaging, and for example a Talbot interferometer using an interference pattern generated by a diffraction grating is known.

The image of phase modulation (phase image) is an image of which contrast represents the distribution of the refractive index of radiation inside an object (difference in refractive angle of radiation), and can visualize the contour of the internal structure. The image of scattering intensity (scattering image) is an image of which contrast the distribution of scattering intensity of radiation inside the object. Where the radiation is scattered while passing through an object, the clarity (also called “visibility”) of the radiation intensity pattern decreases. Information obtained by quantifying the visibility is the scattering image. For example, since radiation scattering intensifies in a portion where fine structures with a size from several microns to several tens of microns are densely aggregated, the scattering image can provide information on the densely aggregated portion or density of the fine structures.

A dynamic range of image data that can be outputted in an imaging apparatus is typically determined in advance, and imaging of an object generating a signal exceeding the dynamic range cannot be performed. Therefore, where imaging of a plurality of objects that differ significantly in characteristics relating to radiation is performed with the same imaging apparatus, good image data can be obtained for some objects, but not for others. For example, in an object in which the scattering intensity is too large, the visibility of intensity pattern decreases as a whole. Therefore, there hardly appears a contrast caused by the presence of fine structures (difference in density). Further, in an object in which the refractive angle is too large, phase wrapping (the phase shifts by +2π) appears in a portion in which the phase change amount exceeds a range from −π to +π, and an artifact called “phase jump” can be generated in the phase image.

Where there are many types of imaging objects or there is a significant spread in object properties, it is not easy to set a dynamic range that ensures good images for all of the object. Therefore, for example, with respect to the phase image, the dynamic range is artificially expanded by image processing performed with software or the like. Japanese Patent Application laid-open No. 2013-42788 discloses a method for correcting a phase jump by unwrapping processing.

However, with the processing for restoring the contrast from low-contrast data or restoring (unwrapping) the wrapped phase, a restoration error can occur or noise can increase, and highly reliable image data are difficult to obtain.

### SUMMARY OF THE INVENTION

Accordingly, it is an object of the present invention to provide a technique that enables imaging in a dynamic range matching an object in a radiation imaging apparatus.

The present invention provides a radiation imaging apparatus of imaging an object by using radiation, the radiation imaging apparatus comprising: a beam splitter; a detector that detects radiation passing through the beam splitter and the object; and a processing device that generates image data representing information on an inside of the object on the basis of data obtained with the detector, wherein a first distance which is a distance between the beam splitter and the object can be changed, and the processing device has a determination unit that determines the first distance that is to be set when the object is imaged, such that a value of information on the inside of the object calculated on the basis of the data obtained with the detector falls within a dynamic range of the image data.

In accordance with the present invention, it is possible to perform imaging in a dynamic range matching an object in a radiation imaging apparatus.

Further features of the present invention will become apparent from the following description of exemplary embodiments with reference to the attached drawings.

### BRIEF DESCRIPTION OF THE DRAWINGS

FIG. 1 is a schematic diagram illustrating a radiation imaging apparatus according to an embodiment of the present invention;

FIG. 2 is a schematic diagram illustrating a radiation imaging apparatus according to the embodiment of the present invention;

FIG. 3 is a schematic diagram illustrating an imaging process according to Example 1 of the present invention;

FIG. 4 is a schematic diagram illustrating an imaging process according to Example 2 of the present invention;

FIG. 5 is a schematic diagram illustrating an imaging process according to Example 3 of the present invention;

FIG. 6 is a schematic diagram illustrating an imaging process according to Example 4 of the present invention;

FIG. 7 is a graph illustrating the relationship between a first distance (d) and scattering information (visibility); and

FIGS. 8A and 8B are schematic diagrams illustrating the relation between the first distance (d) and phase sensitivity.

### DESCRIPTION OF THE EMBODIMENTS

The present invention relates to a radiation imaging apparatus that performs imaging of an object by using radiation such as X-rays, β-rays, and γ-rays, and more particularly to radiation phase imaging for acquiring information (phase information, scattering information, and the like) on the inside of the object on the basis of changes (distortions) of a radiation intensity pattern that has passed through the object. The present invention, can be advantageously used, for example, in a medical imaging apparatus or a nondestructive inspection apparatus. In the case of a medical imaging apparatus, the object is a living body, and the object in the case of a nondestructive inspection apparatus is an inspection object such as an industrial product.

The preferred embodiments of the present invention will be explained hereinbelow in greater detail on the basis of the appended drawings. In the drawings, like members are denoted with like reference numerals and the redundant explanation thereof is herein omitted.

### Embodiment

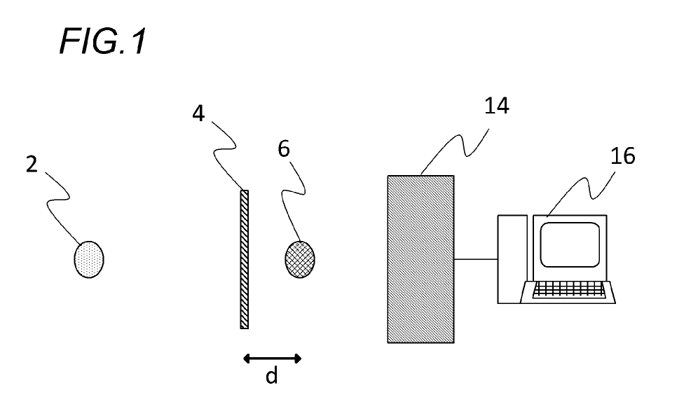

FIG. 1 is a schematic diagram illustrating a radiation imaging apparatus according to an embodiment of the present invention.

The radiation imaging apparatus depicted in FIG. 1 is constituted by a radiation source 2, a beam splitter 4, a detector 14, and a processing device 16. The radiation source 2 may use bremsstrahlung X-rays or characteristic X-rays generated by collision of an electron beam with a target, or X-rays or β-rays generated from a radioactive isotope. The beam splitter 4 is a member having a function of spatially modulating the intensity of radiation generated from the radiation source 2 and forming an intensity pattern having a predetermined spatial period. The beam splitter 4 is, for example, a grating (also called a multi-slit or a multi-pin-hole) in which regions through which radiation can be transmitted (transmitting regions) and regions through which radiation cannot be transmitted (non-transmitting regions) are arranged alternately, or a diffraction grating generating an interference pattern. The detector 14 is a two-dimensional radiation detector in which a plurality of radiation detecting elements are arranged as an array. Two-dimensional image data (radiation intensity image data) representing the spatial distribution of radiation intensity detected by the detector 14 are outputted. The processing device 16 has a function of an image processing device that performs image processing with respect to the image data obtained with the detector 14 and extracts target information or generates image data, and a function of a control device that controls the operation of the entire imaging apparatus.

Where the object 6 is disposed in a radiation propagation path between the radiation source 2 and the detector 14, the radiation is absorbed, refracted, and scattered by the object 6. Therefore, image data obtained with the detector 14 include absorption information, phase information, and scattering information reflecting the internal structure and composition of the object 6. The processing device 16 has a function of extracting the absorption information, phase information, and scattering information from the image data obtained with the detector 14 and generating image data representing the information on the inside of the object 6. The absorption information, that is, the image of which contrast represents the distribution of the absorbance of radiation inside the object, is called absorption image data. The phase information, that is, the image of which contrast represents the distribution of the refractive index of radiation inside the object, is called phase image data. The scattering information, that is, the image of which contrast represents the scattering intensity of radiation inside the object, is called scattering image data. The information extracted by the processing device 16 and the generated image data can be displayed on a display device or outputted to an external computer.

The processing device 16 can be constituted by a computer including a CPU (processing unit), a memory, an auxiliary storage device, an input device, a display device, an input/output interface, and a communication device. Various functions relating to image processing or control of the imaging apparatus are realized by CPU reading and executing a program stored in the auxiliary storage device. The processing device 16 may be constituted by a typical personal computer or an embedded computer. All or some of the functions can be also realized by a circuit such as ASIC or FPGA.

Values calculated as phase information and scattering information are determined by the phase sensitivity determined by the structure of the radiation imaging apparatus. The values to be calculated are typically calculated as values within a certain limited range (dynamic range). Where the calculated values exceed the dynamic range, the values reach saturation and become constant, or wrapping thereof occurs. For example, the absorption information or scattering information reaches saturation at a minimum value or a maximum value, and the phase information wrapping occurs in a range from −π to π.

Accordingly, the radiation imaging apparatus of the present embodiment is configured such that the distance between the object 6 and the beam splitter 4 (referred to as the first distance (d)) can be changed. Where the first distance (d) is changed according to the object 6, the phase sensitivity of the radiation imaging apparatus is adjusted (for example, the phase sensitivity is decreased in the case of an object with a high refractive index or an object with a high scattering intensity). Therefore, the values of phase information or scattering information calculated from the image data obtained with the detector 14 (values of information on the inside of the object) can be controlled to fall within the dynamic range. As a result, the conventional post-processing such as contrast correction or phase unwrapping becomes unnecessary, thereby making it possible to reduce the restoration error and artifacts of the object image. In the radiation imaging apparatus of the present embodiment, the distance between the beam splitter 4 and the detector is fixed.

The first distance (d) may be a dimensionless distance. For example, where the object 6 is located between the beam splitter 4 and the detector 14, the first distance is a value obtained by dividing the distance between the object 6 and the beam splitter 4 by the distance between the beam splitter 4 and the detector 14. In this case, the first distance (d) is a value between 0 and 1.

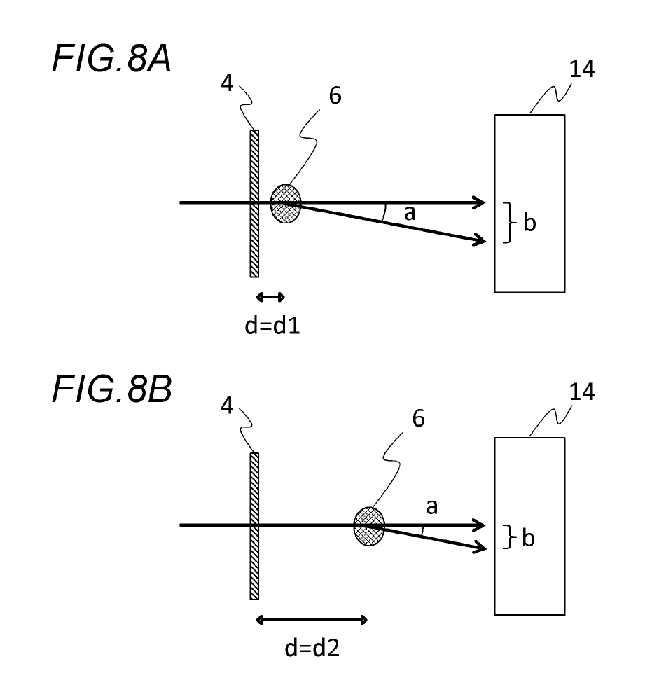

FIGS. 8A and 8B are schematic diagrams illustrating why the phase sensitivity is changed by a change in the first direction (d). FIG. 8A illustrates an example in which the object 6 is disposed at the first distance (d)=d1 between the beam splitter 4 and the detector 14, and FIG. 8B illustrates an example in which the same object 6 is disposed at the first distance (d)=d2 (d1<d2). In the radiation phase imaging, the change between the case in which the object 6 is absent (that is, the radiation propagates linearly) and the case in which the object 6 is present (that is, the radiation is refracted) is regarded as a phase contrast in the detector 14. As follows from FIGS. 8A and 8B, the larger is the distance between the object 6 and the detector 14, the larger is the change (phase contrast) detected by the detector 14. Where a change amount (b) detected by the detector 14 with respect to a refractive angle (a) in the object 6 is called phase sensitivity, the larger is the distance between the object 6 and the detector 14, the higher is the phase sensitivity. Conversely, the smaller is the distance between the object 6 and the detector 14, the smaller is phase sensitivity. In other words, in the arrangement depicted in FIG. 8A, the smaller is the first distance (d), the higher is the phase sensitivity, and the larger is the first distance (d), the smaller is the phase sensitivity.

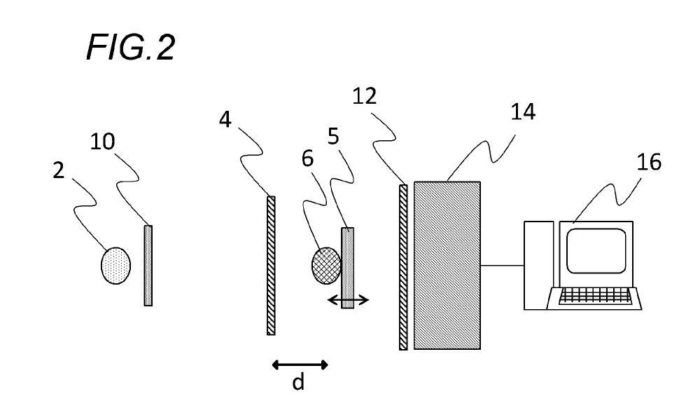

A radiation diffraction grating may be used as the beam splitter 4. Where the transmission region of the beam splitter 4 decreases, the transmitted radiation is spread by the diffraction effect and the intensity modulation is degraded. With the diffraction grating, by using the diffraction effect for spatial intensity modulation, it is possible to obtain good spatial intensity modulation. Further, a transmission-type phase grating may be used as the diffraction grating. Where a transmission-type phase grating is used, the aperture ratio of the beam splitter 4 with respect to radiation becomes 100%, and the radiation utilization efficiency increases. Where the phase grating is used, the distance between the phase grating and the detector may be the so-called Talbot length at which the contrast of interference pattern is high. This type of interferometer is called Talbot interferometer.

Where a diffraction grating is used as the beam splitter 4, an absorption grating 12 may be provided in front of the detector 14 as depicted in FIG. 2. The wavelength of spatial intensity modulation of radiation when a diffraction grating is used can be less than the length of one side of a typical radiation detecting element. In this case, the interference pattern formed by the diffraction grating cannot be resolved by the detector 14. Accordingly, the absorption grating (shielding grating) 12 having a period equal or close to that of the spatial intensity modulation is arranged to generate a moiré with the interference pattern and the absorption grating 12. The moiré corresponds to an image in which the period of interference pattern is enlarged. As a result, imaging of the interference pattern (radiation intensity distribution) can be performed using the detector 14 with a typical resolution.

Further, where a diffraction grating is used as the beam splitter 4, a source grating 10 may be disposed in front of the radiation source 2, as depicted in FIG. 2. To generate interference with the diffraction grating, it is necessary that radiation have spatial coherence. The size of the radiation source 2 and the distance between the radiation source 2 and the diffraction grating are factors that determine the spatial coherence. The size of the radiation source 2 necessary to generate interference with a diffraction element which is less than the detection element of the detector 14 at a distance of 1 m from the radiation source 2 is about several micrometers. Where the focal point of the radiation source 2 is small, the intensity of generated radiation decreases and a long imaging time is needed to obtain an image with a good signal/noise ratio. Accordingly, both the intensity of radiation and spatial coherence can be achieved by using the source grating 10 for the large radiation source 2. This type of interferometer is also a Talbot interferometer, but is specifically referred to as Talbot-Lau interferometer.

In FIGS. 1 and 2, the object 6 is disposed between the beam splitter 4 and the detector 14. The advantage of such a configuration is that even when the first distance (d) is changed, the size of the object image formed on the imaging face of the detector 14 does not change, and therefore an imaging surface area (field of view) can be ensured. Meanwhile, a configuration in which the object 6 is disposed between the radiation source 2 and the beam splitter 4 may be also used (this configuration is not shown in the figure). In this case, a value obtained by dividing the distance between the object 6 and the beam splitter 4 by the distance between the radiation source 2 and the beam splitter 4 is taken as the first distance (d). The advantage of such a configuration is that where the object 6 is disposed between the radiation source 2 and the beam splitter 4, a magnification of the object image can be performed according to the first distance (d). Further, where the object 6 is thus disposed between the radiation source 2 and the beam splitter 4, the smaller is the first distance, the lower is the phase sensitivity.

The first distance (d) may be changed by moving the object 6, by moving the beam splitter 4, or by moving both the object 6 and the beam splitter 4. Where a holding unit that holds and secures the object, such as an object table, is used as the movement unit, the holding unit may be moved automatically (control with the processing device 16) or manually, and where the object 6 is a human, the position or posture of the object 6 itself may be changed. Where manual movement is used, or where the object 6 changes the position or posture for itself, guidance relating to the position which is to be changed or the location to which the object is to be moved may be outputted by text, image, or voice. The output of those types of guiding information is performed by outputting the first distance determined by the processing device 16 to a voice output device (for example, a speaker) or an image display device (for example, a display) connected to the processing device. Where a plurality of lights indicating the position of the object is disposed, guide display may be performed by lighting up the light with the distance to the beam splitter which is the closest to the first distance determined by the processing device 16. In the case of automatic movement, or in the case of manual movement in which the holding unit is moved by manually inputting the movement amount of the holding unit, an instruction from the processing device 16 or a movement amount input unit is outputted to the movement unit. In this case, the movement unit can move the holding unit; for example, an actuator can be used therefor. Where the beam splitter 4 is moved, automatic or manual movement can be used in the same manner as when the holding unit is moved.

In the configuration depicted in FIG. 2, where a diffraction grating is used as the beam splitter 4 and the object 6 is disposed between the beam splitter 4 and the detector 14, the source grating 10 and the beam splitter 4 may be linked by a support member. The object 6 is sometimes difficult to move when adjusting the first distance (d). Where the object 6 is difficult to move, it is necessary to move the beam splitter 4. Where the source grating 10 and the beam splitter 4 are linked by the support member, it is easy to move the beam splitter 4 while maintaining the mutual arrangement of the source grating 10 and the beam splitter 4, and relative positions of the source grating 10 and the beam splitter 4 are not needed to be adjusted. For the same reason, a support member linking the beam splitter 4 and the absorption grating 12 (or the detector 14) may be provided such that the distance between the beam splitter 4 and the absorption grating 12 (or the detector 14) does not change.

The processing device 16 of the present embodiment determines the first distance (d), which should be set when imaging the object 6, on the basis of information relating to the object 6. Any information relating to the object 6 which is to be referred to in order to determine the first distance (d) may be used, provided that this information is correlated with the minimum value, maximum value, or numerical range of information (phase information, scattering information, and the like) on the inside of the object 6.

For example, features extracted from the image data on the object 6 for which imaging has been performed in advance may be used as information relating to the object 6. In this case, pre-imaging may be performed before the main imaging of the object 6, and the information relating to the object 6 may be calculated from the data obtained in pre-imaging. By determining values of information on the object from the pre-imaging data and adjusting the first distance (d) such that the values fall within a dynamic range, it is possible to obtain good image in the main imaging. By performing the pre-imaging at a dose lower than that of the main imaging, it is possible to obtain a good image while reducing the exposure level.

The phase information on the object 6 also may be used as the information relating to the object 6. Where the phase sensitivity of the imaging apparatus is too high with respect to the phase change amount of the object 6, the phase information on the object 6 is calculated over the dynamic range from −π to +π and phase wrapping occurs. For example, by analyzing data of a differential phase image generated on the basis of data obtained in pre-imaging and detecting an artifact caused by phase discontinuity, it is possible to determine whether or not a phase jump is present. Since the phase sensitivity in the case of the configuration depicted in FIG. 2 is inversely proportional to the first distance (d), where the phase sensitivity is suppressed by increasing the first distance (d) and the calculated values fall within the scope of the dynamic range, a phase information distribution which is free from a phase jump can be obtained.

Scattering information (visibility) on the object 6 also may be used as the information relating to the object 6. Where the phase sensitivity of the imaging apparatus is too high with respect to the scattering intensity of the object 6, the calculated value reaches saturation and meaningful difference sometimes is not revealed between the calculated values of scattering intensity (loss of contrast), or the calculated values cannot be distinguished from noise even when density distribution in a microstructure is present inside the object. Accordingly, for example, it is possible to calculate the representative value (average value or the like) of the scattering intensity of the object 6 on the basis of the pre-imaging data and adjust the first distance (d) such as to increase the phase sensitivity when the representative value of the scattering intensity is less than a predetermined value (optimum value) and to decrease the phase sensitivity when the representative value of the scattering intensity is greater than the predetermined value (optimum value).

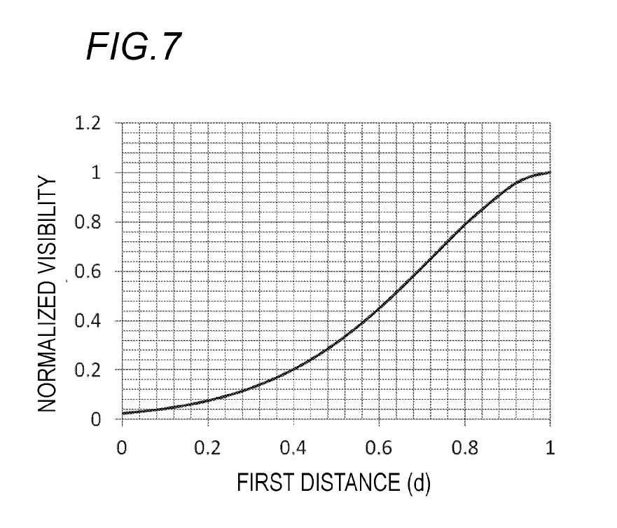

The relationship between the first distance (d) and the scattering intensity can be derived analytically or by computer simulation. For example, FIG. 7 depicts the relationship between the first distance (d) and the scattering intensity in a certain object model. The first distance (d) is plotted against the abscissa, d=0 corresponds to a state in which the object 6 is in contact with the beam splitter 4, and d=1 corresponds to a state in which the object 6 is in contact with the detector 14 (or the absorption grating 12). Plotted against the ordinate is a value V/V0 (normalized visibility) obtained by dividing the value V of visibility of the entire image by the value V0 of visibility of the entire image (background image) in a state in which the object 6 is not present. The value of visibility of the entire image can be calculated by Fourier transforming the entire image and dividing the peak value of the first-order spectrum thereof by the peak value of the zero-order spectrum. The value of normalized visibility corresponds to the average value of scattering intensity of the object 6 and has a negative correlation with the scattering intensity (in order words, where the scattering intensity of the entire object is large, the normalized visibility approaches 0, and where the scattering intensity is small, the normalized visibility approaches 1).

Since the value of local scattering intensity inside the object 6 can be found to be distributed about the average value of the scattering intensity of the entire image as a center, it can be said that the value of normalized visibility may be close to 0.5 (in other words, 0.5 is the optimum value). Meanwhile, with the second configuration, it is clear, as depicted in FIG. 7, that the phase sensitivity deceases and the value of normalized visibility increases as the first distance (d) increased. Accordingly, for example, the value of normalized visibility of the entire image is calculated from the data obtained in pre-imaging, and the first distance (d) is adjusted such that this value approaches 0.5. Thus, where the normalized visibility is less than 0.5, the first distance (d) is increased, and where the normalized visibility is greater than 0.5, the first distance (d) is decreased. By so adjusting the phase sensitivity according to the representative value of scattering intensity, it is possible to prevent the value of scattering intensity of the object 6 from saturation and fit the distribution of scattering intensity in the dynamic range.

Further, information relating to the object 6 may be based on a database. For example, where the object is a human, standard values of the refractive index and the human structure are determined on the basis or test data, or the like, according to the gender, age, or image segment. The first distance (d) such that values of information on the inside of the object calculated when imaging is performed with respect to the object with standard values fall within the dynamic range is determined for each property of the object, such as the gender, age, and imaging segment. Then, the correspondence relationship between the properties of the object (gender, age, and segment) and the first distance (d) is prepared as a database. When the imaging of the object 6 is actually performed, the processing device 16 reads and sets the first distance (d) corresponding to the properties of the object 6 from the database. With such a method, the pre-imaging becomes unnecessary. Therefore, the processing can be accelerated and the exposure dose can be further reduced.

More specific examples of the present embodiment will be explained below with reference to the drawings.

### Example 1

Explained in Example 1 is a radiation imaging apparatus in which an image with good scattering information is obtained using pre-imaging and main imaging.

As depicted in FIG. 2, the radiation imaging apparatus of the present example is constituted by the radiation source 2, the source grating 10, the beam splitter 4, the object table 5, the absorption grating 12, the detector 14, and the processing device 16. All of the constituent components, except for the processing device 16, are disposed on the optical path of the radiation generated from the radiation source 2. The radiation source 2, the source grating 10, the beam splitter 4, the object table 5, the absorption grating 12, and the detector 14 are disposed in the order of description.

For example, X-rays with an energy of 35 keV are used as the radiation. Where X-rays of 35 keV are used, the radiation source 2 can use, for example, rotating anticathode X-rays with tungsten as a target. A computational device such as the so-called workstation or personal computer is used as the processing device 16. The source grating 10 and the absorption grating 12 have a structure in which X-ray transmitting regions and shielding regions are periodically repeated. The transmitting regions are formed from silicon, and the shielding regions are formed from gold. The transmitting regions may be constituted by a light element with a small X-ray attenuation coefficient. The shielding regions may be constituted by a heavy element with a large X-ray attenuation coefficient. Where the shielding regions are a free-standing structure such as a mesh, the transmitting regions may be the voids therein.

A phase grating that ensures phase spatial modulation of the radiation wavefront is used as the beam splitter 4. For example, the phase grating is constituted by a light element with a small X-ray attenuation coefficient and has a structure in which regions with mutually different phase modulation amounts are arranged alternately. In order to obtain a relative difference in the modulation amount, the thickness may be changed while using the same element, or different elements may be used. In the present example, a phase grating is used as the beam splitter 4, but a shielding grating or a lens array may be also used as the beam splitter 4. The beam splitter 4, the source grating 10, and the absorption grating 12 may be fabricated using a MEMS process, or a mechanical processing may be used, or they may be fabricated with a nanoimprinting technique.

The beam splitter 4 generates the interference pattern of X-rays that have been transmitted by the source grating 10. The interference pattern is a set of periodic changes of regions with high and low radiation intensity. Where a diffraction grating is used as the beam splitter 4, the interference pattern is the clearest at the so-called Talbot length determined according to the period of the diffraction grating and the wavelength of the X-rays. The absorption grating 12 is disposed at the Talbot length and the imaging of the interference pattern is performed with the detector 14. Information on the inside of the object 6 is calculated from the interference pattern.

A method for adjusting the first distance (d) is explained hereinbelow. The object 6 is disposed such as to be in contact with the object table 5 disposed between the beam splitter 4 and the absorption grating 12. Where the object 6 is disposed such as to be in intimate contact with the object table 5, the distance between the object 6 and the beam splitter 4, that is, the first distance (d), can be adjusted by changing the position of the object table 5.

The calculation of information on the inside of the object 6 is explained below. The information on the inside of the object 6 can be obtained by processing with the processing device 16 the image data on the interference pattern obtained with the detector 14. For example, the processing of a Fourier transform method is used. A fringe scanning method may be also used. For example, by using the Fourier transform method, various types of information can be calculated from a difference between the interference pattern obtained without the object 6 (undistorted data) and the interference pattern obtained when the object 6 is present (data including distortions caused by the object 6). In the present example, scattering information is calculated from changes in the visibility of the interference pattern. In addition to the scattering information, absorption information may be calculated from changes in the average intensity of the interference pattern and phase information may be calculated from changes in the spatial phase of the interference pattern.

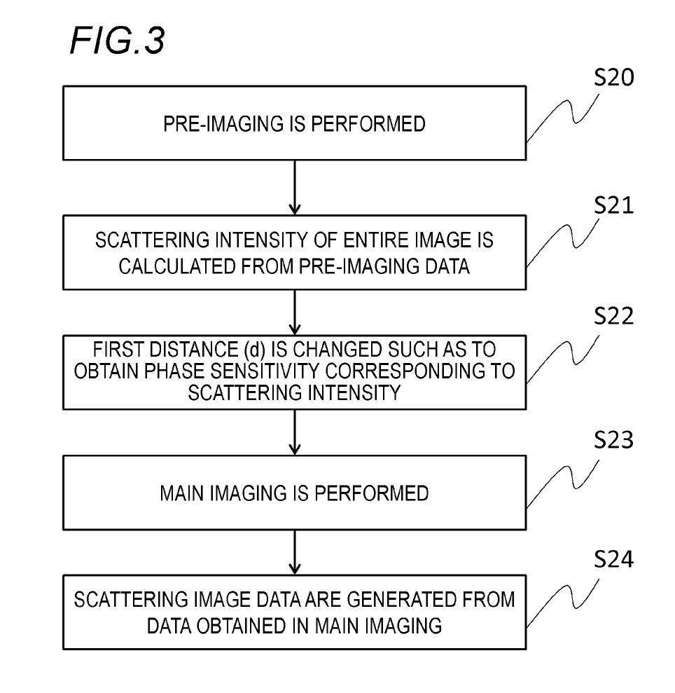

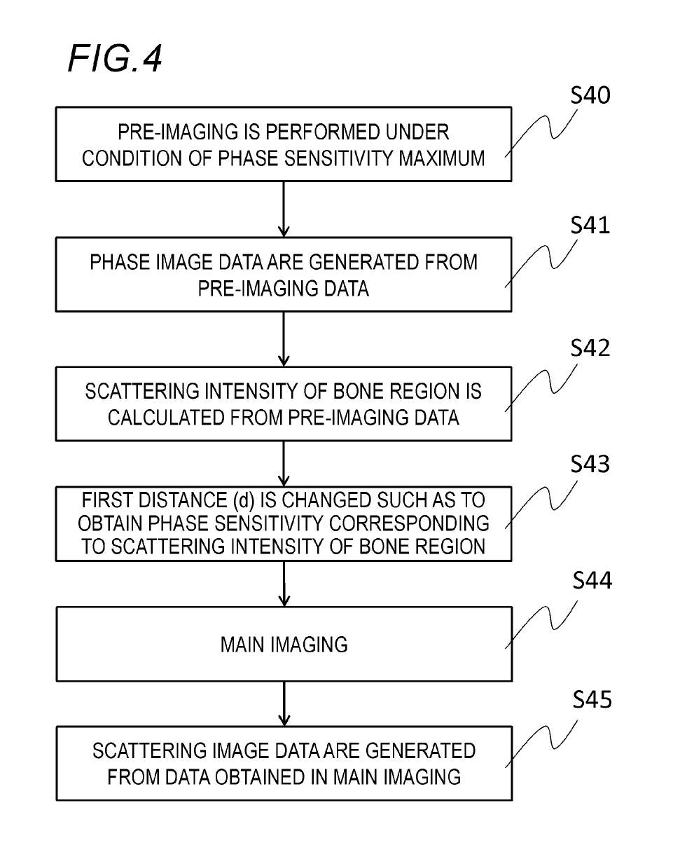

The imaging procedure is explained hereinbelow with reference to FIG. 3. In the present example, the explanation is focused on acquiring scattering information on a human chest. Human lungs are aggregates of alveoli, and the scattering information is obtained as a distribution of scattering intensity caused by the alveoli. The processing operation depicted in FIG. 3 is realized by the processing device 16 which controls each unit of the imaging apparatus and computes the obtained data (same in FIGS. 4 to 6).

Initially, pre-imaging is performed under the control by the processing device 16 at a small tube current such that the exposure dose is 1/10 that during the main imaging (step S20). For example, the imaging is performed at a tube voltage of 80 kV, a tube current of 300 mA, and an exposure time of 40 msec. The image data obtained in pre-imaging are called pre-imaging data. Then, the processing device 16 calculates the normalized visibility representing the average data on scattering intensity of the entire image from the pre-imaging data (step S21). The specific calculation method is described hereinabove. The processing device 16 then changes the first distance (d) to an adequate value such as to obtain the phase sensitivity corresponding to the value of normalized visibility (step S22). Where value of normalized visibility obtained from the pre-imaging data is a predetermined optimum value (for example, 0.4-0.6) and a sufficient contrast is apparently obtained, the phase adjustment of step S22 may be skipped.

The first distance (d) is changed by moving the object table 5. The object table 5 may be moved manually. In this case, the processing device 16 may guide the optimum position of the object table 5 by using text, images, or voice. Where a mechanism for moving the object table 5 is provided, the position of the object table 5 may be automatically adjusted according to the first distance (d) determined by the processing device 16. A length measuring device prepared in advance may be used for positioning the object 6 or the object table 5. For example, where a length measuring device using a laser is used, the length can be measured without coming into contact with the beam splitter 4, and the object 6 or the object table 5 can be positioned with good accuracy. The first distance (d) may be also visualized by disposing a position indicator provided with a scale, such as a ruler, in the movable range of the object table 5.

After the object table 5 has been moved, the main imaging is performed at an exposure time of 400 milliseconds (step S23). The processing device 16 uses the Fourier transform method to generate the scattering information (scattering image) on the object from the data obtained in the main imaging (step S24). With the above-described processing, it is possible to obtain good scattering image data while suppressing the exposure of the object.

In the present example, the imaging method using scattering information is explained, but phase information may be also used. In Example 3, an imaging method using phase information is explained. Each information may be used individually, as in the present example, or composite information may be used which is obtained by computing two or more types of information. The information in this case includes any two types of information selected from phase information, absorption information, and scattering information. For example, in the scattering information, the contrast is generated on the basis on different principles between the contour of the object 6 and the non-contour region. Where imaging of scattering information on the non-contour region of the object 6 is desired, it is desirable that the image of the scattering information be optimized by the value of the non-contour region. Accordingly, absorption information and scattering information are used, and the effect of the contour of the object 6 is removed from the scattering information by the differential absorption information obtained by extracting the contour of the object 6 by spatial differentiation of the absorption information, thereby making it possible to handle the obtained information as the scattering information on the non-contour region of the object 6. The contour information may use the absorption information or may be determined by using the phase information.

Further, the object table is used in the present example, but a bed may be used instead of the object table. In this case, the bed may be moved, or the radiation imaging apparatus may be moved relative to the secured bed. Where the object 6 maintains the position thereof for a certain time, it is possible not to use the table. The certain time referred to herein means, for example, the time required for imaging.

### Example 2

Example 2 is different from Example 1 in that information on different regions of interest of the object 6 is obtained by pre-imaging and main imaging. The configuration of the radiation imaging apparatus is the same as in Example 1 and the explanation thereof is herein omitted.

In the present example, the imaging procedure is explained by considering the case in which phase information and scattering information on a human hand is acquired. A human hand includes as imaging objects a soft tissue region constituted by the so-called soft tissue including ligaments, tendons, and cartilage, and a bone region constituted by hard bones. The phase information is suitable for imaging the soft tissue region, and the scattering information is suitable for imaging the bone region. However, the optimum phase sensitivity is not necessarily the same for the phase information and scattering information.

As depicted in FIG. 4, initially, pre-imaging of the soft tissue region is performed by minimizing the first distance (d), that is, maximizing the phase sensitivity (step S40). The processing device 16 generates phase information (phase image data) from the pre-imaging data (step S41). Where the maximum phase sensitivity is sufficient for acquiring information on the soft tissue, the phase information on the soft tissue region can be obtained. Meanwhile, for the bone region, since the cancellous body included in the bones generally demonstrates large scattering, scattering information on the bone region cannot be obtained in the optimum dynamic range in an apparatus in which the phase sensitivity is too high. Accordingly, the processing device 16 calculates the representative value (normalized visibility) of scattering intensity of the bone region from the per-imaging data (step S42), calculates the first distance (d) optimum for obtaining the scattering information on the bone region on the basis of the representative value, and adjusts the position of the object 6 (step S43). Then, the main imaging is performed at the first distance (d) after the adjustment (step S44). Scattering image data are generated on the basis of the data obtained in the main imaging (step S45), thereby making it possible to acquire scattering image data in which the structure of the bone region is clearly visualized.

The phase image data obtained in step S41 and the scattering image data obtained in step S45 may be displayed side by side or displayed by switching on the display device. Alternatively, the processing device 16 may generate, on the basis of the two types of image data, combined image data in which the soft tissue region is visualized with the phase information and the bone region is visualized with the scattering information, and the combined image data may be displayed on the display device.

In the present example, the phase information on the soft tissue region is acquired by pre-imaging, but the scattering information on the soft tissue region may be also acquired. In some cases, microcalcination groups are formed by mineralization inside the soft tissue. Since those groups can be obtained as scattering information, phase information may be acquired from the main imaging by performing the imaging of microcalcination groups inside the soft tissue region in pre-imaging which is set to the first distance (d) different from that of the main imaging. The information obtained in the pre-imaging and main imaging may be of one type or a plurality of types. An image which is the best for the radiation imaging apparatus is acquired by performing optimization with respect to the region of interest which is the object of each imaging operation.

### Example 3

Example 3 is different from Example 1 in that phase information which free from a phase jump is obtained by using pre-imaging and main imaging. The configuration of the radiation imaging apparatus is the same as in Example 1 and the explanation thereof is herein omitted.

In the present example, the phase information on the object 6 is calculated by pre-imaging. The phase information obtained with the Talbot interferometer is represented in the image as the phase change of the interference pattern. The changes in phase are typically wrapped between −π and π. Therefore, where the calculated value of phase information exceeds π, the phase changes abruptly from π to −π, that is, the so-called phase jump occurs, even in the case of a continuous phase change.

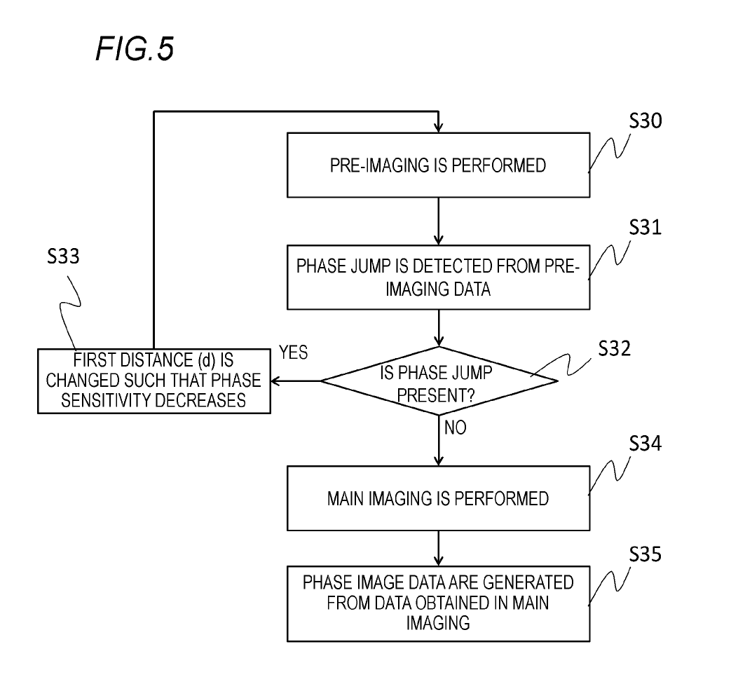

Accordingly, in the present example, as depicted in FIG. 5, initially, pre-imaging is performed (step S30), and then the processing device 16 detects a phase jump by using the pre-imaging data (step S31). The phase jump may be detected by any method. For example, the phase image data generated from the pre-imaging data are compared with the absorption image data (or scattering image data), and it is examined whether an edge included in the phase image data also appears in the absorption image data (or scattering image data). Where a feature such as the edge does not appear in the absorption image data (or scattering image data), this feature is highly probable to be an artifact caused by the phase jump.

Where it is determined that the phase jump is present (YES in step S32), the processing device 16 changes the first distance (d) such that the phase sensitivity decreases (step S33). For example, with the configuration depicted in FIG. 2, the first distance (d) is increased by a predetermined amount (for example, 0.2). The pre-imaging is then performed again (step S30), and the presence/absence of the phase jump is determined (steps S31, S32). The optimum first distance (d) is determined such as to obtain the phase sensitivity which is free of the phase jump by repeatedly determining the presence/absence of the phase jump and changing the first distance (d). The main imaging is performed (step S34) after a state without the phase jump has been reached (NO in step S32). In the data obtained in the main imaging, the phase change falls within the dynamic range (from −π to +π). Therefore, phase image data which are free from a phase jump can be generated (step S35).

### Example 4

Example 4 differs from Example 1 in that the first distance (d) in the main imaging is determined on the basis of a database. The configuration of the radiation imaging apparatus is the same as in Example 1 and the explanation thereof is herein omitted.

In the present example, the case of imaging a human chest is explained. A database in which the characteristic parameters of a human body (more specifically, gender, age, weight, height) and the corresponding optimum first distance (d) are associated with each other is prepared in advance. The database may be stored in the internal storage device of the processing device 16 or in an external storage device or other server. The optimum first distance (d) may be determined by simulation or on the basis of clinical test data.

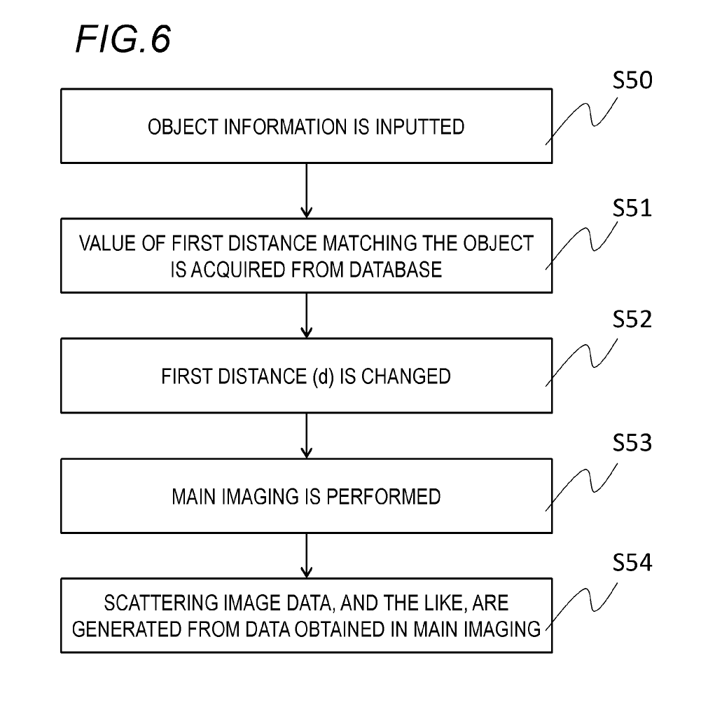

As depicted in FIG. 6, an operator of the radiation imaging apparatus inputs information on the gender, age, weight, and height of the object 6 into the processing device 16 (step S50). The processing device 16 acquires the best-matching value of the first distance (d) on the basis of the inputted information on the object 6 by referring to the database (step S51). Then, the processing device 16 controls the object table 5 according to the first distance (d) determined in step S51 and adjusts the position of the object 6 (step S52). The main imaging is then performed (step S53), and scattering image data and/or phase image data are generated on the basis of the data obtained in the main imaging (step S54). With such a method, adequate data acquisition can be performed in the same manner as in Examples 1 to 3. Further, since no pre-imaging is required in the in the present example, the exposure dose can be reduced by comparison with those in Examples 1 to 3.

In the present example, a human body is considered as the object 6, but the object 6 may be other than the human body. The parameters in the database are also not limited to the gender, age, weight, and height, and for example shape, volume, and material may be used as parameters. Some or all of the parameters may be used when determining the first distance (d).

The examples described hereinabove are merely the preferred specific example of the present invention and are not intended to limit the scope of the present invention. The present invention can assume a variety of specific forms within the scope of the technical concept thereof. For example, the processing operations described in Examples 1 to 4 can be combined together.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-098835, filed on May 12, 2014, which is hereby incorporated by reference herein in its entirety.

What is claimed is:

1. A radiation imaging apparatus of imaging an object by using radiation,
the radiation imaging apparatus comprising:
a beam splitter;
a detector that detects radiation passing through the beam splitter and the object; and
a processing device that generates image data representing information on an inside of the object on the basis of data obtained with the detector, wherein
a first distance which is a distance between the beam splitter and the object can be changed, and
the processing device has a determination unit that determines the first distance that is to be set when the object is imaged, such that a value of the information on the inside of the object falls within a dynamic range of the image data.

2. The radiation imaging apparatus according to claim 1, further comprising:
a movement unit configured to move a position of the beam splitter or the object, or positions of both the beam splitter and the object according to the first distance determined by the determination unit.

3. The radiation imaging apparatus according to claim 2, further comprising:
a holding unit that holds the object, wherein
the movement unit moves the holding unit.

4. The radiation imaging apparatus according to claim 1, wherein
the determination unit determines the first distance that is to be set when main imaging of the object is performed, by using pre-imaging data on the object which have been obtained by pre-imaging.

5. The radiation imaging apparatus according to claim 4, wherein
the pre-imaging of the object is performed at a dose lower than that of the main imaging.

6. The radiation imaging apparatus according to claim 4, wherein
the determination unit calculates a representative value of a scattering intensity of the object from the pre-imaging data, and determines the first distance such that a phase sensitivity is higher than that of the pre-imaging when the representative value is less than a predetermined value, and such that the phase sensitivity is lower than that of the pre-imaging when the representative value is greater than the predetermined value.

7. The radiation imaging apparatus according to claim 4, wherein
the determination unit generates phase image data on the object from the pre-imaging data, and when a phase jump is included in the phase image data, determines the first distance such that a phase sensitivity is lower than that of the pre-imaging.

8. The radiation imaging apparatus according to claim 4, wherein
the determination unit generates phase image data on the object from the pre-imaging data and generates scattering information on the object from the main imaging data, or generates scattering image data on the object from the pre-imaging data and generates phase information on the object from the main imaging data.

9. The radiation imaging apparatus according to claim 1, wherein
the determination unit determines the first distance that is to be set when main imaging of the object is performed, by referring to a database in which the first distance is associated with each property of the object.

10. The radiation imaging apparatus according to claim 1, wherein
the beam splitter is a phase grating, and a distance between the phase grating and the detector is set to a Talbot length.

11. The radiation imaging apparatus according to claim 10, further comprising
an absorption grating disposed in front of the detector.

12. The radiation imaging apparatus according to claim 1, further comprising
a source grating disposed between a radiation source that generates radiation and the beam splitter.

13. The radiation imaging apparatus according to claim 12, further comprising
a support unit that links the source grating and the beam splitter such that a distance between the source grating and the beam splitter does not change.

14. The radiation imaging apparatus according to claim 1, wherein
the imaging is performed by disposing the object between the beam splitter and the detector.

15. The radiation imaging apparatus according to claim 1, wherein
the imaging is performed by disposing the object between a radiation source that generates radiation and the beam splitter.

16. The radiation imaging apparatus according to claim 1, wherein
the processing device comprises a display device, and
the display device displays an image based on the image data.

17. The radiation imaging apparatus according to claim 4, wherein
the processing device comprises a display device, and
the display device displays image data generated from the pre-imaging data and image data generated from the main imaging.

18. The radiation imaging apparatus according to claim 17, wherein
the display device displays a combined image of image data generated from the pre-imaging data and image data generated from the main imaging.
